(19) United States
(12) Patent Application Publication
(10) Pub. No.: US 2021/0307994 A1
(43) Pub. Date: Oct. 7, 2021

(54) EYE EXERCISE DEVICE

(71) Applicant: Byoung Hak LEE, Incheon (KR)

(72) Inventor: Byoung Hak LEE, Incheon (KR)

(21) Appl. No.: 17/287,546

(22) PCT Filed: Nov. 18, 2019
(86) PCT No.: PCT/KR2019/015736

§ 371 Date: Apr. 22, 2021

(30) Foreign Application Priority Data
Nov. 20, 2018 (KR) 10-2018-0143245
Nov. 20, 2018 (KR) 10-2019-0092698
Jul. 30, 2019 (KR) 10-2019-0092699

(51) Int. Cl. A61H 5/00

(52) U.S. Cl. CPC A61H 5/00; A61H 2201/0188; A61H 2201/1607

(57) ABSTRACT

An eye exercise device includes: a barrel which is worn on a face and shields a wearer's eyes; and forward gaze plates which are disposed so as to face the wearer's eyes on one side of the barrel, wherein the forward gaze plates are provided with at least one of a throughhole for guiding light provided from the outside of the barrel to the wearer's eyes and an LED for irradiating illumination light toward the wearer's eyes.

### TECHNICAL FIELD

The present invention relates to an eye exercise device configured to induce ocular exercise of a wearer using light emitting diodes (LED).

### BACKGROUND ART

Due to a variety of visual media, eye health of modern people is currently under threat. Accordingly, people are more aware of health and are recommended to frequently do a variety of eyeball exercises to relieve eye fatigue.

Accordingly, exercise devices configured to improve visual acuity have been developed. As one of such exercise devices configured to improve visual acuity, “Personal eye care system” is disclosed in Korean Patent Publication No. 10-2010-0035742 (Apr. 7, 2010).

The personal eye care system disclosed as the related art includes a glasses-type body 1, a lens 5 provided in the body 1, and acupressure rods installed around the lens to face a wearer's face.

Also, a shutter is installed in the body 1 to allow the wearer to do exercise of relaxing and contracting irises of the wearer by forming light and shade in the body 1 according to the shutter being opened or closed when the wearer gazes forward while wearing the eye care system.

Also, light emitting diodes (LEDs) are installed on an inner periphery of the lens 5 provided in the body 1 so that the wearer gazes at the blinking LEDs to allow the eyeballs of the wearer to trace the LED which emits light and to naturally use muscles connected to the eyeballs so as to allow the wearer to do exercise of assisting visual acuity improvement.

However, in the ocular muscle exercise device according to the related art, since positions of the LEDs installed on the inner periphery of the lens 5 and blinking are not suitable for eyeball exercise for each type such as myopia, hyperopia, or the like, even when eyeball exercise is performed using the same, it is actually impossible to improve (restore) visual acuity.

That is, in the ocular muscle exercise device according to the related art, since the LEDs installed on the inner periphery of the lens 5 are only circularly arranged causing a blinking order of the LEDs to be limited to being circular, musculus rectus superior oculi, musculus obliquus superior oculi, external rectus, musculus obliquus inferior oculi, musculus rectus inferior bulbi, medial rectur, and the like which move eyeballs are not evenly exercised and do not significantly assist visual acuity improvement.

Also, since the ocular muscle exercise device according to the related art does not consider eye exercise for improving astigmatism, astigmatic people or amblyopic people with astigmatism do not receive any help in visual acuity improvement.

### DISCLOSURE

### Technical Problem

The present invention is directed to providing an eye exercise device configured to assist visual acuity improvement by exercising irises to adjust a pupil size of a wearer and relaxing and contracting a variety of muscles such as musculus rectus superior oculi, musculus obliquus superior oculi, external rectus, musculus obliquus inferior oculi, musculus rectus inferior bulbi, medial rectur, and the like which adjust movement of eyeballs.

### Technical Solution

One aspect of the present invention provides an eye exercise device including a body tube worn on a face to shield eyes of a wearer and a forward gaze plate disposed on one side of the body tube to face the eyes of the wearer. Here, the forward gaze plate includes at least any one of a through hole configured to guide light provided from an outside of the body tube to the eyes of the wearer and a light emitting diode (LED) configured to provide lighting light toward the eyes of the wearer.

The eye exercise device may further include a front pin hole portion provided in the forward gaze plate and configured to guide external light to the through hole formed in the forward gaze plate.

A front holder provided on the forward gaze plate and including a space portion therein and a front pin hole through which external light flows and a scattering prevention portion provided in the front holder to induce linearity of light passing through the front pin hole may be included.

The scattering prevention portion may include at least any one of a lighting guide embedded in the front holder and including an optical path configured to allow the light flowing in through the front pin hole to pass therethrough or a lens member configured to diffuse or focus the light flowing in through the front pin hole.

The lighting guide may be embedded in the front holder and include at least any one of a disk and a block body in which the optical path is formed.

The scattering prevention portion may include a plurality of such lighting guides in which the optical path is provided, and the lighting guides may be embedded in the front holder while the respective optical paths communicate with each other.

At least any one of the lighting guides may include a single groove portion or a plurality of groove portions formed in an inner circumferential surface, on which the optical path is formed, and configured to prevent sideward-diffused light of incident light flowing in through the optical path from being irregularly reflected so that the diffused light is guided along the optical path.

The lighting guide may include at least one disk and at least one block body, in which the optical paths are formed, and be embedded in the front holder while the disk and the block body are overlapped with each other, and the block body and the disk may be embedded in the front holder while the respective optical paths communicate with each other.

The lens member may be embedded in the front holder while being overlapped with the lighting guide.

The eye exercise device may further include an indirect lighting device configured to contract or expand a pupil of the wearer by providing blinking lighting light separate from light passing through the front pin hole to at least one of the optical paths of the lighting guides configured to allow the light flowing in through an inside of the front holder or the front pin hole to pass therethrough.

The forward gaze plate may include a main blinking module which is provided in an inner surface thereof facing the wearer and in which a plurality of such LEDs are spaced apart from each other and configured to blink the LEDs in a pattern set to induce a gaze of the wearer to sequentially provide lighting light to the eyes of the wearer.

The forward gaze plate may include a through hole formed in a center of the inner surface and may guide light provided from an outside of the body tube to the eyes of the wearer separately from the LEDs, and the LEDs may be arranged radially or on all sides on the basis of the through hole and provide lighting light based on the light from the outside which is emitted through the through hole.

A pair of such forward gaze plates may be provided, and the body tube may further include a forward movement portion configured to move the pair of forward gaze plates to dispose the LEDs or the through holes at positions corresponding to the eyes of the wearer.

The forward movement portion may include a rack and pinion gear or a bevel gear to move the forward gaze plates and move the forward gaze plates as the forward gaze plates are connected to some components and in connection therewith.

The eye exercise device may further include a sideward gaze plate installed on a side of the forward gaze plate and including an LED configured to provide lighting light toward the eyes of the wearer or a through hole configured to guide light from an outside of the body tube to the eyes of the wearer to induce a gaze of the wearer sideward.

The sideward gaze plate may further include a side pin hole portion configured to guide the light provided from the outside of the body tube to the through hole.

The side pin hole portion may include a side holder including a side pin hole, through which the light provided from the outside of the body tube flows in, and a space portion provided therein, and a scattering prevention portion provided on the side holder and configured to induce linearity of the light passing through the side pin hole.

The scattering prevention portion may include at least any one of a lighting guide embedded in the side holder and including an optical path configured to allow the light flowing in through the side pin hole to pass therethrough or a lens member configured to diffuse or focus the light flowing in through the side pin hole.

The sideward gaze plate may include an auxiliary blinking module which is provided in an inner surface thereof facing the wearer and in which a plurality of such LEDs are spaced apart from each other and configured to blink the LEDs in a pattern set to induce a gaze of the wearer sideward to sequentially provide lighting light to a side of the wearer.

The sideward gaze plate may include a through hole formed in a center of the inner surface and guides light provided from an outside of the body tube to the eyes of the wearer separately from the LEDs, and the LEDs may be arranged radially or on all sides on the basis of the through hole and provide lighting light based on the light from the outside which is emitted through the through hole.

The sideward gaze plate may be moved along a sidewall of the body tube by a sideward movement portion provided in the body tube.

The sideward gaze plates may be formed at an angle tilted from the forward gaze plate and gradually move farther away from each other from a front of the body tube toward the wearer.

The eye exercise device may further include a rear lighting device configured to provide lighting separate from the LED of the forward gaze plate to a rear of the forward gaze plate.

The rear lighting device may include a rear LED configured to operate separately from the LED of the forward gaze plate and to emit rear lighting to the rear of the forward gaze plate, a cap configured to shade the rear LED, and a gaze hole formed in the cap to pass the lighting of the rear LED therethrough.

The eye exercise device may further include a bush installed in the through hole of the forward gaze plate to guide light flowing in through the through hole straight toward a rear of the forward gaze plate.

### Advantageous Effects

Since an eye exercise device according to one embodiment of the present invention has a structure in which lenses for improving myopia, hyperopia, and astigmatism are provided in a lens holder and a structure in which a plurality of light emitting diodes (LEDs) configured to induce periocular muscles to adequately move are arranged on a gaze plate, the eyes of the wearer may be moved according to the blinking LEDs and may relax and contract overall muscles configured to move eyeballs so as to improve visual acuity of the wearer.

Also, in the eye exercise device according to one embodiment of the present invention, a lighting device is installed to be adjacent to the gaze plate and the wearer's iris reacts to blinking of the lighting device and performs pupil exercise so as to improve visual acuity of the wearer.

Also, since the eye exercise device according to one embodiment of the present invention has a structure configured to move a lens corresponding to a distance between the eyes of the wearer, the device may be applied to a variety of users while being not limited to a particular wearer so as to assist visual acuity improvement.

Also, in the eye exercise device according to the present invention, positions of a front pin hole portion and a side pin hole portion may vary so as to be applied to a variety of users while being not limited to a particular wearer so as to assist visual acuity improvement.

### DESCRIPTION OF DRAWINGS

FIG. 1 is a perspective view of an eye exercise device of the present invention.

FIG. 2 is a plan cross-sectional view of the eye exercise device of the present invention when viewed from above.

FIG. 3 is a coupled cross-sectional view illustrating components of a pin hole portion of the eye exercise device according to the present invention.

FIG. 4 is a separate cross-sectional view illustrating the pin hole portion shown in FIG. 3.

FIG. 5 is a cross-sectional view illustrating components of a lens member according to the present invention.

FIG. 6 is a perspective view illustrating components of a forward movement portion according to the present invention.

FIG. 7 is a side perspective view illustrating components of the forward movement portion according to the present invention when viewed from the side.

FIG. 8 is a view illustrating a relation of arrangement between a main blinking module and an auxiliary blinking module according to the present invention.

FIG. 9 is a cross-sectional view illustrating components of a pin hole portion according to another embodiment of the present invention.

FIG. 10 is a perspective view illustrating an eye exercise device according to another embodiment of the present invention.

FIG. 11 is a plan cross-sectional view of the eye exercise device shown in FIG. 10 when viewed from above.

FIG. 12 shows views (A)-(E) illustrating another example of a scattering prevention portion of the eye exercise device according to the present invention.

FIGS. 13 and 14 are cross-sectional views illustrating a rear lighting device of the eye exercise device according to the present invention.

FIG. 15 shows views (A)-(E) illustrating still another example of the scattering prevention portion of the eye exercise device according to the present invention.

FIG. 16 shows cross-sectional views (A)-(B) illustrating application of the lens member according to hyperopia and myopia in the eye exercise device according to the present invention.

FIG. 17 shows cross-sectional views (A)-(B) illustrating eye exercise performed by the rear lighting device in the eye exercise device according to the present invention.

FIG. 18 is a concept view illustrating an example of applying a convex lens and a concave lens according to hyperopia and myopia.

FIGS. 19 and 20 are concept views illustrating application of the lens members on a front holder and a side holder in the eye exercise device according to the present invention.

FIG. 21 is a perspective view illustrating components of a forward movement portion according to another embodiment of the present invention.

FIG. 22 is a perspective view illustrating components of a forward movement portion according to still another embodiment of the present invention.

### MODES OF THE INVENTION

The advantages and features of the present invention and a method of achieving the same will become apparent with reference to the attached drawings and following embodiments which will be described below in detail.

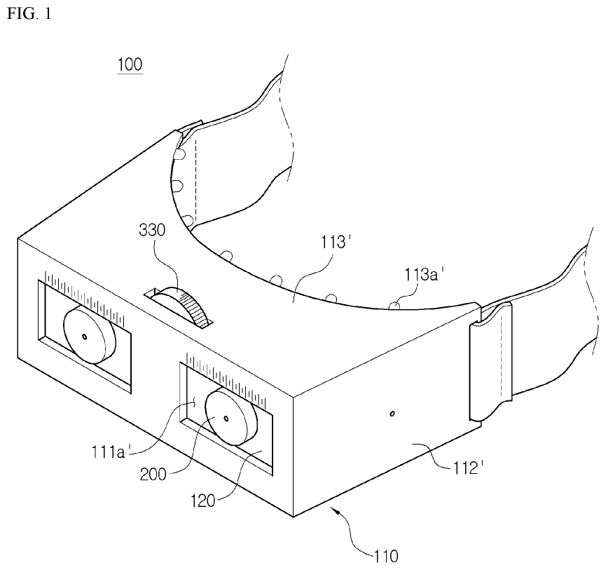

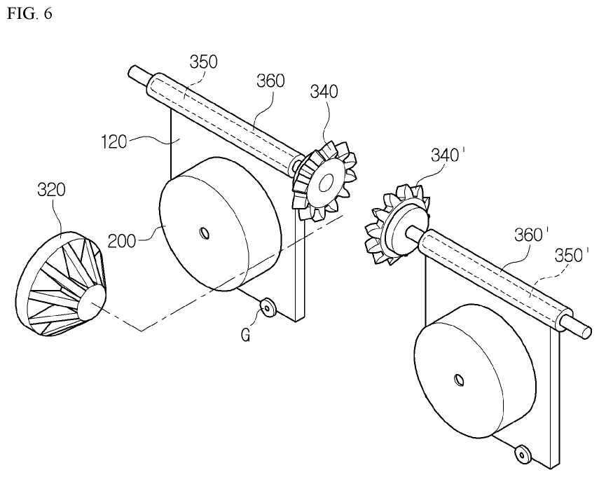

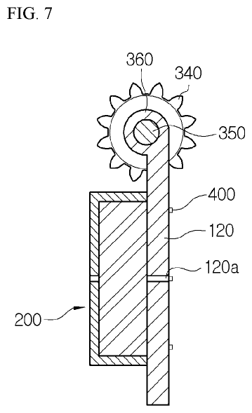

Hereinafter, an eye exercise device according to one embodiment of the present invention will be described in detail with reference to FIGS. 1 to 8. In a description of the present invention, a detailed description of well-known functions or components of the related art will be omitted not to obscure understanding of the embodiments of the present invention.

As shown in FIG. 1, an eye exercise device 100 according to one embodiment of the present invention includes a body tube 110 covering a facial part around eyes of a wearer, a pair of forward gaze plates 120 provided inside the body tube 110 and disposed to face the wearer's eyes on one longitudinal side of the body tube 110, and a front pin hole portion 200 provided in the forward gaze plate 120 to guide external light to through holes formed in the forward gaze plates 120.

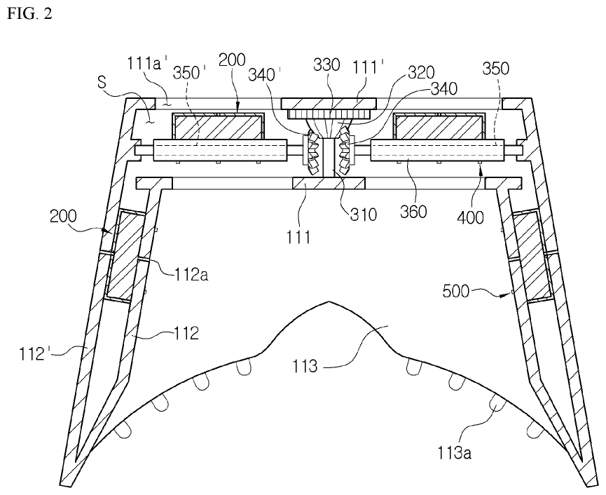

The body tube 110 may have an impregnated body covering a facial part of a wearer and having one open side as shown in FIGS. 1 and 2. That is, the body tube 110 may include a pair of front walls 111 and 111′ disposed in front of the wearer's eyes while being spaced at a certain interval apart from each other, sidewalls 112 and 112′ extending from both lateral ends of the front walls 111 and 111′ toward the wearer and blocking the wearer's lateral sight, and blocking walls 113 and 113′ provided at top and bottom ends of the front walls 111 and 111′ and the sidewalls 112 and 112′ to block external light.

Here, the forward gaze plates 120 and a forward movement portion 300 which will be described below may be disposed in a space portion S formed between the pair of front walls 111 and 111′ of the body tube 110. That is, the forward gaze plates 120 may be provided in the space portion S formed between the pair of front walls 111 and 111′. Also, the forward movement portion 300 that is a device configured to allow the forward gaze plate 120 to be movable in a longitudinal direction of the front walls 111 and 111′ (a direction of a distance between the wearer's eyes) in the space portion S may be provided.

In addition, in one front wall 111 of the pair of front walls 111 and 111′ which is disposed close to the wearer, an opening portion 111a configured to expose the forward gaze plate 120 disposed in the space portion S may be provided. In the other front wall 111′, an opening portion 111a′ configured to expose the front pin hole portion 200 provided in the forward gaze plate 120 may be provided.

Also, a plurality of protrusions 113a and 113a′ configured to perform acupressure on periocular muscles of the wearer when the wearer wears the body tube 110 may be provided on longitudinal ends of the blocking walls 113 and 113′.

The pair of forward gaze plates 120 are components disposed to face the eyes of the wearer who wears the body tube 110 so that the wearer may see one surface of the forward gaze plate 120 through the opening portion 111a formed in the one front wall 111.

Also, the front pin hole portion 200 is provided in the other surface of the forward gaze plate 120 so that the front pin hole portion 200 may be exposed outward through the opening portion 111a′ formed in the other front wall 111′ of the body tube 110.

Accordingly, the wearer who wears the body tube 110 and sees the forward gaze plate 120 may receive light flowing in through a through hole 120a of the forward gaze plate 120 via the front pin hole portion 200.

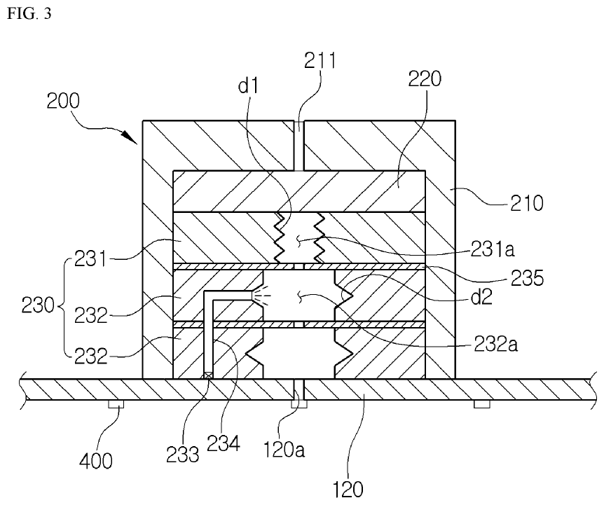

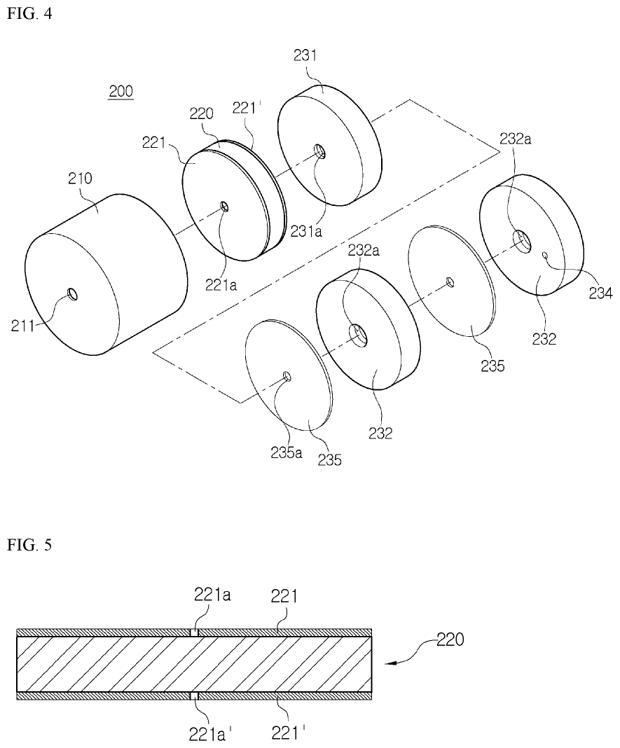

As shown in FIGS. 3 and 4, the front pin hole portion 200 may include a front holder 210 in which a space portion is provided and which has one side with a front pin hole 211 through which external light flows in and formed thereon and the other side connected to the forward gaze plate 120, a lens member 220 provided in the space portion of the front holder 210, and a scattering prevention portion 230 configured to induce linearity of light which has passed through the lens member 220.

Here, the scattering prevention portion 230 may be configured as at least one of a light guide embedded in the front holder 210 and including an optical path configured to pass light flowing in through the front pin hole 211 and the lens member 220 configured to diffuse or focus light flowing in through the front pin hole 211.

Also, the lighting guide of the scattering prevention portion 230 may be configured as at least one of a block body or disk which is embedded in the front holder 210 and includes an optical path.

The front holder 210 may have a cylindrical shape having one side blocked and the other side open and may include the one side in which the front pin hole 211 through which external light flows in is provided as described above. Also, the other side of the front holder 210 may be open to allow a variety of components included in the lens member 220 and the scattering prevention portion 230 to be inserted into an internal space portion formed by the front holder 210.

Here, the scattering prevention portion 230 includes at least one block body which provides an optical path through which light having passed through the front pin hole 211 flows in and is embedded in the front holder 210. Also, a plurality of such block bodies provided in the scattering prevention portion 230 are provided, overlapped while the optical paths face each other, and embedded in the front holder 210.

In addition, the longitudinal other side of the front holder 210 is connected to the other surface of the forward gaze plate 120. In the drawings of the present invention, the longitudinal other side of the front holder 210 is shown as coming into simple contact with the other surface of the forward gaze plate 120 but the present invention is not limited thereto and an annular groove may be formed in the other surface of the forward gaze plate 120 to allow the longitudinal other side of the front holder 210 to be inserted into the forward gaze plate 120 using a forcible fitting method. Accordingly, the front holder 210 may be detachably coupled to the forward gaze plate 120.

A case is provided so that the forward gaze plate 120 may be embedded in the case. The front holder 210 and a cap 131 of a rear lighting device 130 which will be described below may be integrally provided in the case. The case may include a lighting hole at a position corresponding to a light emitting diode (LED) of a main blinking module 400 or 400′ provided on the forward gaze plate 120, which will be described below, to transmit lighting light emitted from the LED.

On the other hand, the case may be configured so that the LED of the main blinking module 400 or 400′ is fixed to a position of the lighting hole or is inserted into and fixed to the lighting hole. In this case, the lighting hole may be omitted as necessary. Also, the case may be configured as one cap having both a part where the lighting hole is formed and a part where the cap 131 is formed that are formed convexly or may be configured as a single cap or a plurality of stepwise caps.

The lens member 220 may be a component inserted from the other side of the front holder 210 and disposed on one longitudinal side of the front holder 210 and may be implemented as a convex lens or a concave lens corresponding to an ocular state of the wearer. For example, the lens member may be implemented as a concave lens corresponding to the ocular state of the wearer who has myopia or myopic amblyopia. On the other hand, the lens member may be implemented as a convex lens corresponding to the ocular state of the wearer who has hyperopia or hyperopic amblyopia.

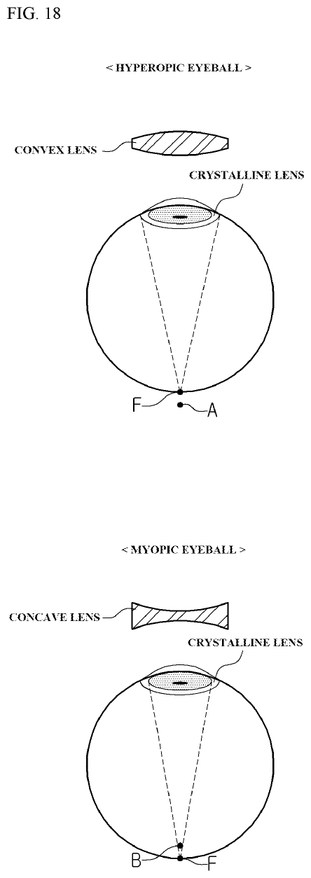

That is, as shown in FIG. 18, since the wearer who has hyperopia has an eyeball or a crystalline lens formed to be longer laterally, the eyeball or crystalline lens is formed to be thin and wide in comparison to a normal eyeball or lens so that a focal point is formed at an outside A of retina. Accordingly, when a farsighted person wears convex-lens glasses, like the convex lens shown in FIG. 18, a focal point is formed at a normal focal point F of retina so that focal movement may be smoothly performed.

Also, as shown in FIG. 18, since the wearer who has myopia has an eyeball or a crystalline lens formed to be longer longitudinally, the eyeball or crystalline lens is formed to be thick and narrow in comparison to a normal eyeball or lens so that a focal point is formed at an inside B of retina. Accordingly, when a shortsighted person wears concave-lens glasses, like the concave lens shown in FIG. 18, a focal point is formed at a normal focal point F of retina so that focal movement may be smoothly performed.

Meanwhile, as shown in FIG. 5, one surface or the other surface of the lens member 220 may be coated with a shading sheet 221 or 221′ including a pin hole 221a or 221a′. The shading sheet 221 or 221′ allows external light flowing in through the front pin hole 211 formed in the front holder 210 to be emitted only toward a central portion of the lens member 220 so as to allow the wearer who sees the forward gaze plate 120 to easily receive light through the through hole 120a of the forward gaze plate 120. Here, the pin hole 221a or 221′a formed in the shading sheet 221 or 221′ is disposed at a position corresponding to the through hole 120a formed in the forward gaze plate 120.

Accordingly, the external light may be easily delivered through the through hole 120a of the forward gaze plate 120 by the shading sheets 221 and 221′ placed on both surfaces of the lens member 220. For reference, although one embodiment of the present invention in which both surfaces of the lens member 220 are coated with the shading sheets 221 and 221′ has been described, the present invention is not limited thereto. Only one surface of both surfaces of the lens member 220 may be coated with the shading sheet 221 or 221′ and the lens member 220 may be ultraviolet (UV)-coated to protect the surfaces and to block UV rays.

Due to the above component, a pupil size of the wearer who wears the body tube 110 and sees an inner surface of the forward gaze plate 120 may increase or decrease by gazing at emitted light passing through the lens member 220 and the through hole 120a of the forward gaze plate 120. That is, irises of the wearer may be moved by light emitted through the through hole 120a.

As shown in FIGS. 3 and 4, the scattering prevention portion 230 includes a disk 235 in which a through hole transmitting light of the front pin hole 211 of the front holder 210 or an optical path of the block body is formed and which is overlapped with at least one of the front holder 210 or the block body. Here, the front holder 210 may further include the lens member 220 embedded in with the scattering prevention portion 230 and configured to diffuse or focus light flowing into the front pin hole 211.

In addition, a first block body 231 includes a first optical path 231a through which light passing through the lens member 220 flows in and a plurality of second block bodies 232 including second optical paths 232a connected to the first optical path 231a formed on the first block body 231 to communicate therewith.

The first block body 231 and the second block bodies 232 may have a shape corresponding to a cross section of the space portion formed by the front holder 210 to be accommodated in the front holder 210. For example, when the cross section of the space portion formed in the front holder 210 is a circle, the first block body 231 and the second block body 232 may have a disciform or cylindrical shape.

The first block body 231 may be inserted from the other open side of the front holder 210 and be pressed against the lens member 220. Also, the second block body 232 may be inserted from the other open side of the front holder 210 and be pressed against the first block body 231.

The first block body 231 and the second block bodies 232 perform a function of preventing external light sequentially passing through the front pin hole 211 provided in the front holder 210 and the lens member 220 from being scattered. That is, light passing through the front pin hole 211 provided in the front holder 210 and the lens member 220 may be allowed to be transmitted with linearity to the eyes of the wearer who wears the body tube 110.

The first optical path 231a provided in the first block body 231 may include a plurality of screw grooves d1. That is, a screw groove d1 may be formed along an entire inner circumferential surface of the first block body 231 dividing the first optical path 231a.

Accordingly, diagonally scattered light of light passing through the lens member 220 and flowing into the first optical path 231a may flow into a plurality of such screw grooves d1 formed in the first optical path 231a so that only light having linearity may be moved through the first optical path 231a and transmitted to the second optical path 232a of the second block body 232.

That is, the light having passed through the lens member 220 may be prevented from being scattered by the first block body 231 primarily. Also, one or two screw grooves d2 may be provided in the second optical path 232a provided in the second block body 232. That is, the screw groove d2 may be formed in only a part of an inner circumferential surface of the second block body 232 dividing the second optical path 232a.

The second block body 232 performs a function of preventing the light primarily scattered by the first block body 231 from being scattered secondarily. Accordingly, it is unnecessary to form the screw groove d2, which is formed in the second block body 232, along the entire inner circumferential surface of the second block body 232.

The plurality of second block bodies 232 may be arranged on the front holder 210. In one embodiment of the present invention, as shown in FIGS. 3 and 4, the two second block bodies 232 are arranged behind the first block body 231.

Accordingly, since light sequentially passing through the front pin hole 211 of the front holder 210 and the lens member 220 is transmitted to the wearer's eyes sequentially via the first optical path 231a of the first block body 231 and the second optical path 232a of the second block body 232, the wearer may receive light having linearity instead of the light scattered by the screw groove d1 formed in the first optical path 231a and the screw groove d2 formed in the second optical path 232a.

Accordingly, the wearer's pupils do not react with the scattered light and react with the light having linearity so that a degree of fatigue applied to the eyes may be reduced.

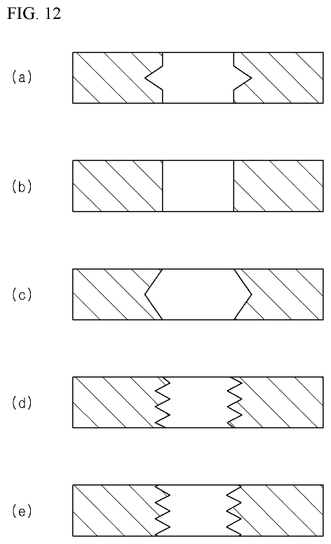

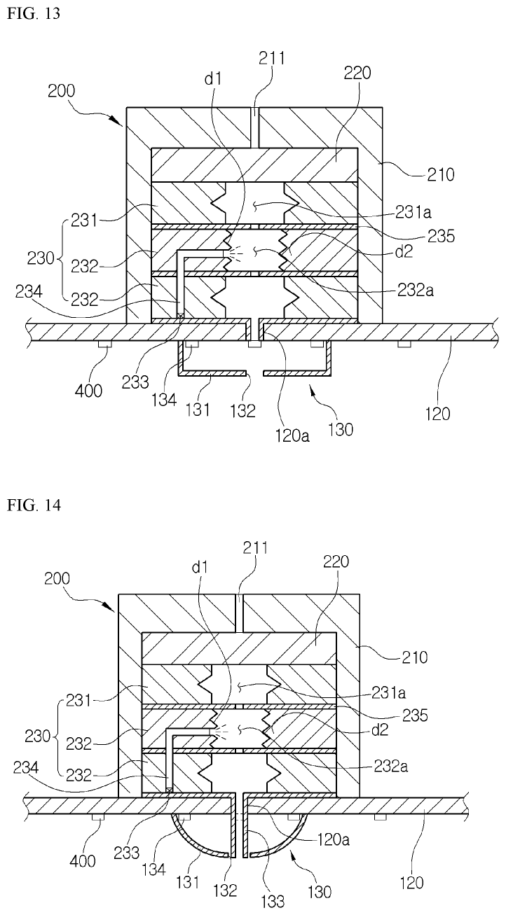

That is, although it has been described above that a plurality of groove portions d1 are formed in the first optical path 231a of the first block body 231 and one or two groove portions d2 are formed in the second optical path 232a of the second block body 232 as shown in FIG. 3, a first block body 231 or second block bodies 232 having shapes shown in FIGS. 12A to 12E may be combined in a variety of shapes as shown in FIGS. 13 and 14 so as to form the scattering prevention portion 230 as necessary.

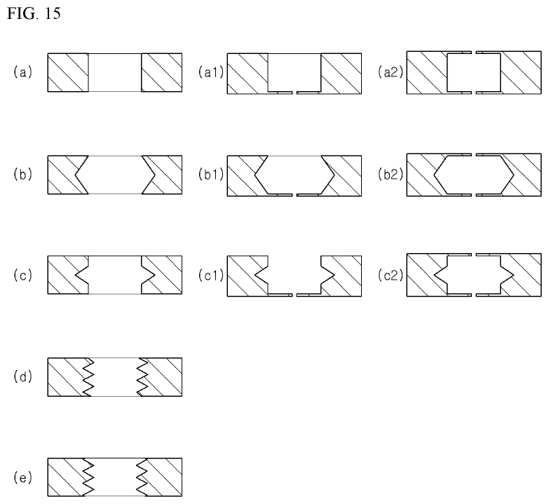

Also, as necessary, the scattering prevention portion 230 may be combined as shown in FIGS. 15A to 15E. Also, the scattering prevention portion 230 may include the first block body 231 and the second block bodies 232 provided repetitively on the front holder 210.

Meanwhile, the scattering prevention portion 230 may include an indirect lighting device 233 provided between the second block bodies 232 and the forward gaze plate 120 and a third optical path 234 configured to guide light emitted from the indirect lighting device 233 to the second optical path 232a and provided in the second block bodies 232.

The indirect lighting device 233 may blink in a certain pattern and transmit lighting light to the wearer so that the lighting light brighter than the light having passed through the lens member 220 may be provided to the wearer.

Accordingly, the pupils of the wearer who gazes at the forward gaze plate 120 may be reduced or dilated instantaneously by the indirect lighting device 233 blinking in a certain pattern so that pupil constriction and relaxation operate to a great extent and exercise effects increase.

The third optical path 234 provides a space in which the indirect lighting device 233 is placeable and the lighting light generated by the indirect lighting device 233 is guidable to the second optical path 232a divided by the second block bodies 232.

The third optical path 234 may be formed to have a variety of shapes and may be formed in one second block body 232 or the plurality of second block bodies 232.

Accordingly, the light generated by the indirect lighting device 233 may flow into the second optical path 232a divided by the second block bodies 232 via the third optical path 234 and may be delivered to the wearer and the wearer gazes at the light having passed through the lens member 220 and the pupils are instantaneously reduced or dilated by blinking of the indirect lighting device 233 so that pupil constriction and relaxation operate to an even greater extent and exercise effects increase.

Meanwhile, the scattering prevention portion 230 may further include a plurality of such disks 235. The disks 235 may divide the first optical path 231a interposed between the first block body 231 and the second block bodies 232 and formed by the first block body 231 from the second optical path 232a formed by the second block bodies 232 and may minimize scattering of light having passed through the first optical path 231a when flowing into the second optical path 232a. Accordingly, a through hole 235a through which light passes may be formed in central parts of the disks 235.

Also, when the plurality of second block bodies 232 are provided in the space portion of the holder 210, the disks 235 may be interposed between one second block body 232 and another second block body 232.

That is, the disks 235 may divide the second optical path 232a formed by one second body 232 from the second optical path 232a formed by another second block body 232 and may minimize scattering of the light having passed through one second optical path 232a when flowing into another second optical path 232a.

As shown in FIGS. 15A1, 15A2, 15B1, 15B2, 15C1, and 15C2, the disks 235 may be provided on one surface of the block body or both surfaces of the block body. Here, the disks 235 may be attached to one surface or both surfaces of the block body or may be integrally molded with the block body.

Meanwhile, as shown in FIGS. 2, 6, and 7, the eye exercise device 100 according to the present invention may further include the forward movement portion 300 configured to move the pair of forward gaze plates 120 to dispose the front pin hole portions 200 at positions corresponding to the wearer's eyes.

The forward movement portion 300 may move the pair of forward gaze plates 120 to be closer to each other or to move to be farther away from each other in the space portion S formed between the pair of front walls 111 and 111′ of the body tube 110.

That is, the pair of forward gaze plates 120 disposed on both sides on the basis of a longitudinal center of the front walls 111 and 111′ are allowed to be movable to be closer to each other toward the center or to be farther away from each other.

The forward movement portion 300 described above may include a first rotating shaft 310 rotatably provided in the body tube 110, a driving bevel gear 320 provided on a longitudinal end of the first rotating shaft 310, a dial 330 provided on the driving bevel gear 320, driven bevel gears 340 and 340′ rotated in connection with rotational movement of the dial 330 and engaged with the driving bevel gear 320, second rotating shafts 350 and 350′ disposed in a direction alternating with a longitudinal direction of the first rotating shaft 310, and rotated in connection with rotational movement of the driven bevel gears 340 and 340′ and transfer members 360 and 360′ configured to be transferable along a longitudinal direction of the second rotating shafts 350 and 350′ and to support the forward gaze plates 120.

Both longitudinal ends of the first rotating shaft 310 may be rotatably provided on the pair of front walls 111 and 111′, respectively. Also, the driving bevel gear 320 may be fixedly connected to the first rotating shaft 310.

The dial 330 is a component rotated by the wearer and may be fixedly connected to the driving bevel gear 320. Here, a part of the dial 330 may be exposed upward from a blocking wall 113′ included in the body tube 110 as shown in FIG. 2. Accordingly, the wearer may rotate the dial 330 exposed above the blocking wall 113′.

The driven bevel gears 340 and 340′ may be fixedly connected to one longitudinal end of the second rotating shaft 350, may be provided as a pair, and may be disposed on both sides of the driving bevel gear 320.

The pair of second rotating shafts 350 and 350′ may be provided in the space portion S, and the driven bevel gears 340 and 340′ may be fixedly provided on the one longitudinal end of thereof as described above. Also, the other longitudinal end thereof may be rotatably connected to the sidewall 112′ of the body tube 110.

Accordingly, when the driving bevel gear 320 is rotated with the dial 330, the pair of driven bevel gears 340 and 340′ may be rotated in opposite directions and the pair of second rotating shafts 350 may also be rotated in opposite directions in connection therewith.

The transfer members 360 and 360′ may have a tubular shape and may be provided on the pair of second rotating shafts 350 and 350′, respectively. Here, the transfer members 360 and 360′ may be connected to the second rotating shafts 350 and 350′ while accommodating the second rotating shafts 350 and 350′.

Here, screw processing portions which are screw-coupled to each other may be formed on inner circumferential surfaces of the transfer members 360 and 360′ and outer circumferential surfaces of the second rotating shafts 350 and 350′ so that the transfer members 360 and 360′ may linearly reciprocate along a longitudinal direction of the second rotating shafts 350 and 350′ when the second rotating shafts 350 and 350′ are rotated.

Accordingly, since the transfer members 360 and 360′ provided on the pair of second rotating shafts 350 and 350′ are movable toward one side or the other side due to rotation of the dial 330 in one direction or another direction, the forward gaze plates 120 provided on the transfer members 360 and 360′, respectively, may be transferred in a direction of becoming closer to each other or a direction of moving away from each other corresponding to a distance between the eyes of the wearer.

In other words, due to the forward movement portion 300, the wearer may move the through holes 120a formed in the pair of forward gaze plates 120 to positions corresponding to left/right pupil positions of the wearer by rotating the dial 330.

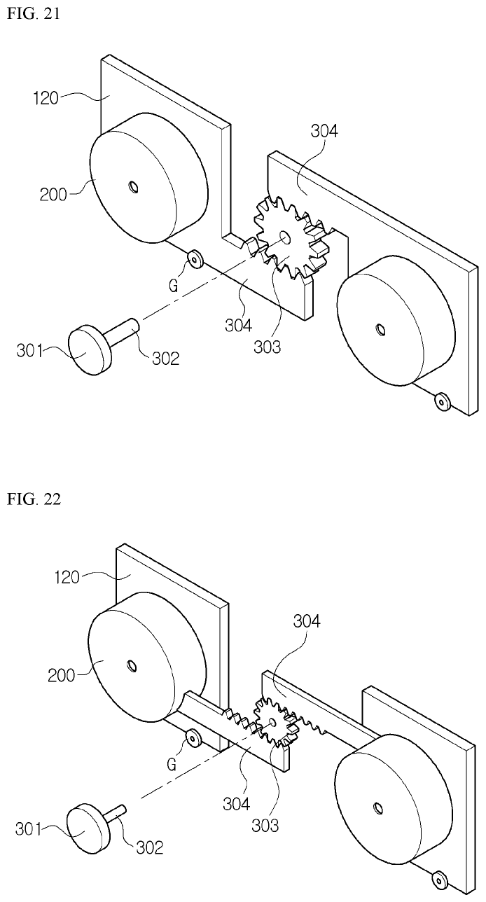

Meanwhile, the above-described forward movement portion 300 is not limited thereto and may be modified. That is, as shown in FIG. 21, the forward movement portion 300 includes a front knob 301, a rotating shaft 302, a first pinion 303, and a first rack 304. The front knob 301 is exposed in front of the body tube and rotatably operated by the wearer. A plurality of uneven parts are formed on an outer circumferential surface of the front knob 301 to allow the wearer to easily adjust the front knob 301 so that sliding may be prevented while the wearer rotatably operates the front knob 301 with a finger.

The front knob 301 is provided on a front surface of the body tube 110, and the rotating shaft 302 extending into the body tube 110 and integrally connected to the front knob 301 is provided on the other end of the front knob 301. Here, the rotating shaft 302 is located between the pair of forward gaze plates 120 so that the rotating shaft 302 is rotated as the front knob 301 is rotatably operated by the wearer.

Also, a first pinion 303 is provided on one longitudinal end of the rotating shaft 302 so that the first pinion 303 is rotated according to rotation of the rotating shaft 302. Also, the first pinion 303 is engaged with the first rack 304 extending from the forward gaze plate 120 so that the pair of forward gaze plates 120 move to be closer to each other or move to be farther away from each other according to rotation of the front knob 301.

Here, the first racks 304 extending from the pair of forward gaze plates 120 are located vertically on the basis of the first pinion 303 and engaged with the first pinion 303.

Due to the above components, according to rotational operation of the front knob 301 provided in front of the body tube 110, the through holes 120a formed in the pair of forward gaze plates 120 may be moved to positions corresponding to the left/right pupil positions of the wearer.

Meanwhile, in the forward movement portion 300, the first rack 304 may be formed in the forward gaze plate 120 as described above and, on a case-by-case basis, may be formed on the front holder 210 included in the front pin hole portion 200 and be engaged with the first pinion 303 as shown in FIG. 22. That is, the front holder 210 is integrally formed with the forward gaze plate 120 and the first rack 304 is formed on the front holder 210.

Also, the first rack 304 formed on the front holder 210 as described above is engaged with the first pinion 303 so that the pair of forward gaze plates 120 move to be closer to each other and to move to be farther away from each other according to the rotation of the front knob 301.

Meanwhile, a guide member G configured to guide the forward gaze plate 120 while the pair of forward gaze plates 120 are moved by the forward movement portion 300 may be provided in a space formed between the front wall 111 and the sidewall 114.

The guide member G may be provided at a top end or a bottom end of the forward gaze plate 120 and may come into sliding contact with the blocking walls 113 and 113′ so that the forward gaze plate 120 may move smoothly while movement generated during the movement of the forward gaze plate 120 may be minimized.

The guide member G may have a well-known pulley shape or a rail shape having a staple-shaped cross section in which the top end or bottom end of the forward gaze plate 120 may be inserted to come into sliding contact therewith.

Also, gradations to allow the wearer to easily see a movement distance of the front pin hole portion 200 may be shown on an outer surface of the front wall 111′ of the body tube 110. Accordingly, the wearer may move the front pin hole portion 200 and the forward gaze plates 120 to correspond to a distance between the left/right pupils of a user by rotating the dial 330 using the gradations before wearing the body tube 110.

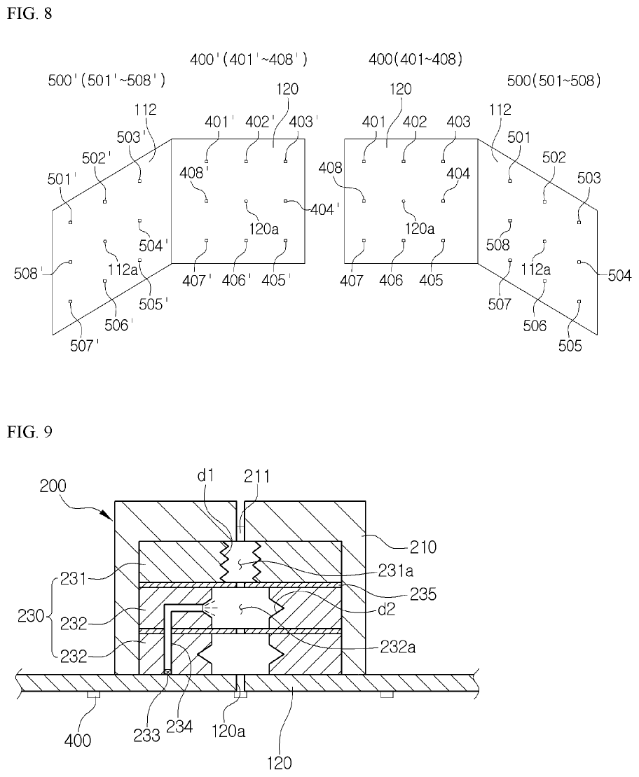

Meanwhile, as shown in FIG. 8, main blinking modules 400 and 400′ configured to blink in the certain pattern to induce a forward gaze of the wearer may be provided on inner surfaces of the forward gaze plates 120.

The main blinking modules 400 and 400′ may include a plurality of LEDs 401 to 408 and 401′ to 408′ arranged radially while being spaced at certain intervals apart from each other on the basis of the through holes 120a formed in the forward gaze plates 120.

In addition, auxiliary blinking modules 500 and 500′ configured to blink in a certain pattern with the main blinking modules 400 and 400′ and to induce a lateral gaze of the wearer may be provided on an inner surface of the sidewall 112 of the body tube 110.

The auxiliary blinking modules 500 and 500′ may also include a plurality of LEDs 501 to 508 and 501′ to 508′ arranged radially while being spaced at certain intervals apart from each other on the basis of through holes 112a formed in the sidewall 112.

Here, the sidewalls 112 may extend, at a tilted angle, from both longitudinal ends of the front wall 111 to allow the wearer to see light blinking from the auxiliary blinking modules 500 and 500′ when the wearer turns his or her gaze laterally. That is, a pair of such sidewalls 112 connected to longitudinal ends of the front wall 111 may extend to be tilted so that a distance between the pair of sidewalls 112 gradually increases toward the wearer.

Meanwhile, the main blinking modules 400 and 400′ and the auxiliary blinking modules 500 and 500′ may be controlled by a control portion which is not shown. That is, the control portion may include a power supply portion configured to supply power to the LEDs 401 to 408 and 401′ to 408′ of the main blinking modules 400 and 400′ and the LEDs 501 to 508 and 501′ to 508′ of the auxiliary blinking modules 500 and 500′ and may include a printed circuit board (PCB) configured to allow a plurality of LEDs to blink in a certain pattern.

For reference, since a technique of supplying power to the LEDs 401 to 408 and 401′ to 408′ of the main blinking modules 400 and 400′ and the LEDs 501 to 508 and 501′ to 508′ of the auxiliary blinking modules 500 and 500′ and controlling blinking thereof is a well-known technical configuration easily performed by those skilled in the art, a detailed description thereof will be omitted not to obscure the essential of the present invention in the specification of the present invention.

Accordingly, the wearer who wears the body tube 110 may perform eye exercise according to the plurality of LEDs 401 to 408 and 401′ to 408′ which blink in a certain pattern on the inner surface of the forward gaze plate 120 and the plurality of LEDs 501 to 508 and 501′ to 508′ which blink in a certain pattern on an inner surface of the sidewall 112 of the body tube 110 on the basis of external light flowing in through the through hole 120a of the forward gaze plate 120 through the front pin hole portion 200.

That is, the wearer may primarily see the external light flowing in through the through hole 120a of the forward gaze plate 120 and see the LEDs 401 to 408 and 401′ to 408′ of the main blinking modules 400 and 400′ and the LEDs 501 to 508 and 501′ to 508′ of the auxiliary blinking modules 500 and 500′ while wearing the body tube 110.

Also, as shown in FIG. 9, in the front holder 210 included in the front pin hole portion 200, the lens member 220 may be omitted and only the first block body 231 and the second block body 232 of the scattering prevention portion 230 may be disposed.

In addition, positions of the lens member 220, the first block body 231, and the second block body 232 which are provided on the front holder 210 may vary. For example, the first block body 231 or the second block body 232 may be disposed at a position closest to the front pin hole 211 of the front holder 210 and then the lens member 220 may be disposed next.

Meanwhile, the eye exercise device according to the present invention includes a sideward gaze plate 630 including at least one of LEDs on the sidewall of the body tube to provide light to the inside of the body tube and through holes configured to guide light provided from an outside of the sidewall of the body tube to the wearer's eyes.

Here, the sideward gaze plate 630 may be configured as an inner sidewall facing the sidewall 112 inside the body tube or configured as a plate-shaped member separately formed from the inner sidewall like the forward gaze plate 120 to be movably installable inside the body tube 110.

The sideward gaze plate 630 includes a side pin hole portion 600 configured to guide light outside the sidewall of the body tube 110 to the through hole formed in the sideward gaze plate 630 and a sideward movement portion 700 configured to move the side pin hole portion 600 according to manipulation of the wearer so as to assist eye exercise of the wearer.

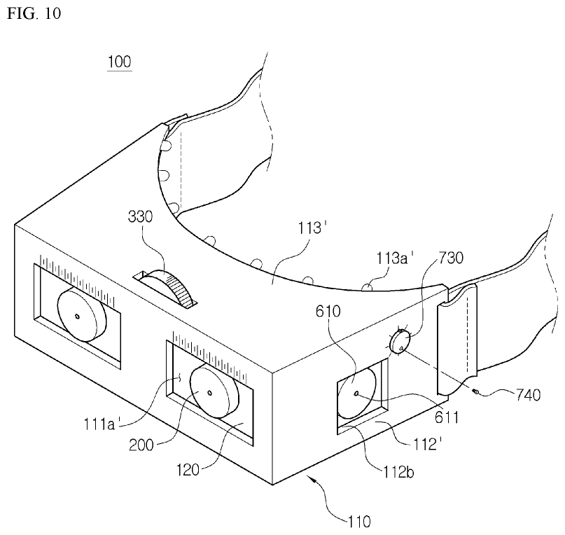

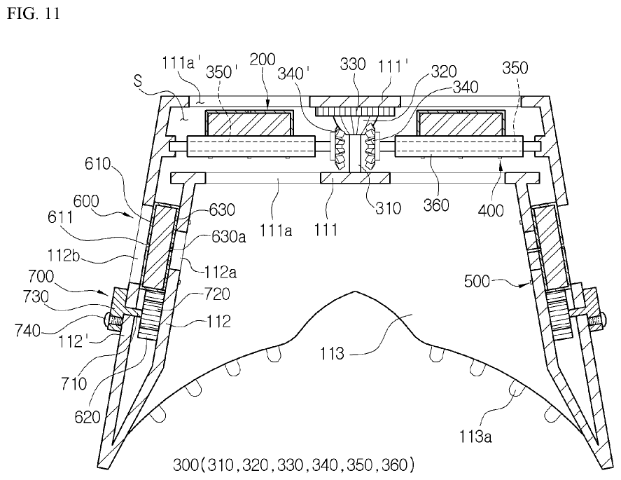

In addition, as shown in FIGS. 10 and 11, the side pin hole portion 600 is provided in a long hole 112b formed in an outer surface 112′ and a side opening portion 112a formed in an inner surface 112 of the sidewall.

The side pin hole portion 600 has a shape equal or similar to that of the front pin hole portion 200 described above. That is, the side pin hole portion 600 has a space portion therein and includes a side holder 610 including a side pin hole 611 through which external light flows in and the scattering prevention portion 230 provided in the side holder 610 on one side. Also, the side pin hole portion 600 may include the above-described indirect lighting device of the front pin hole portion 200.

That is, a case is provided so that the sideward gaze plate 630 may be embedded in the case. The side holder 610 and a cap of the rear lighting device 130 which will be described below may be integrally provided in the case. The case may include lighting holes at positions corresponding to the LEDs of the auxiliary blinking module 500 or 500′ provided on the sideward gaze plate 630, which will be described below, to transmit lighting light emitted from the LEDs.

On the other hand, the case may be configured so that the LEDs of the auxiliary blinking modules 500 and 500′ are fixed to a position of the lighting hole or are inserted into or fixed to the lighting holes. In this case, the lighting holes may be omitted as necessary. Also, the case may be configured as one cap having both a part where the lighting hole is formed and a part where the cap 131 is formed that are formed convexly or may be configured as a single cap or a plurality of stepwise caps.

Here, the lens member 220 of the side pin hole portion 600 may be formed as a concave lens when the lens member 220 of the front pin hole portion 200 provided in the forward gaze plate 120 is formed as a convex lens, and on the other hand, may be formed as a convex lens when the lens member 220 of the front pin hole portion 200 provided in the forward gaze plate 120 is formed as a concave lens.

A reason to form the lens member 220 of the front pin hole portion 200 provided in the forward gaze plate 120 and the lens member 220 of the side pin hole portion 600 provided in the sidewall 112 as different-shaped lenses as described above is to perform exercises of relaxing and contracting a crystalline lens by alternately gazing, by the wearer wearing the body tube 110, at the through hole 120a formed in the forward gaze plate 120 and the side pin hole 611 formed in the side holder 610.

Accordingly, the wearer may turn his or her gaze laterally to look at light flowing into the side pin hole 611 due to the side pin hole portion 600 and perform crystalline lens exercise with iris exercise.

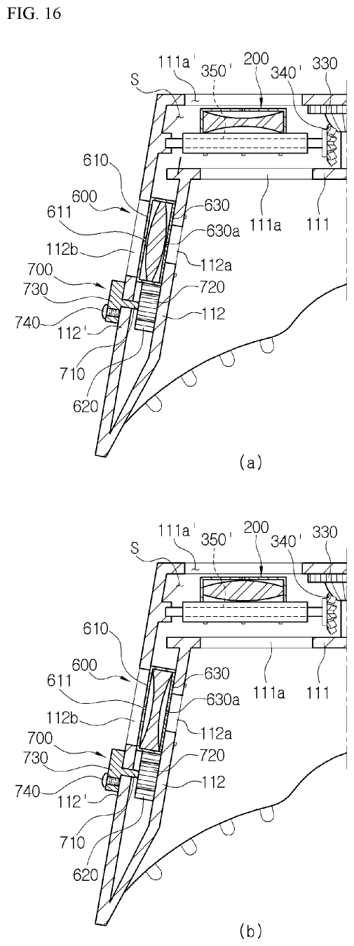

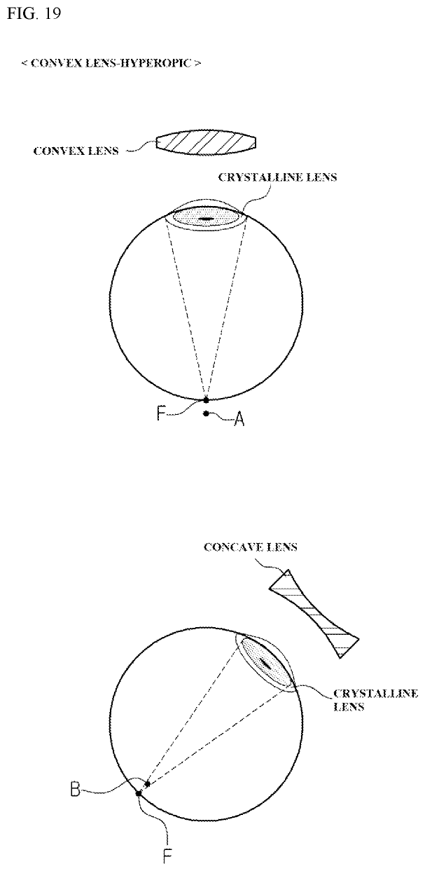

In detail, as shown in FIGS. 16A and 19, when the lens member 220 of the front pin hole portion 200 is a convex lens and the lens member 220 of the side pin hole portion 600 is a concave lens to be configured as a hyperopic type, the convex lens in front allows a focal point of incident light to be formed at a normal focal point F.

On the other hand, when an eyeball is rotated sideward maximally and looks at the concave lens on a side, the eyeball and/or crystalline lens is formed to be slightly longer longitudinally like a myopic eyeball or crystalline lens due to a change in intraocular pressure due to ocular muscle exercise so that a focal point is formed on the inside B of retina. Here, the concave lens of the side pin hole portion 600 lengthens a focal point to form the focal point at the normal focal point F of retina so that the focal point is accurately formed on the retina in the eyeball to smoothly perform focal movement.

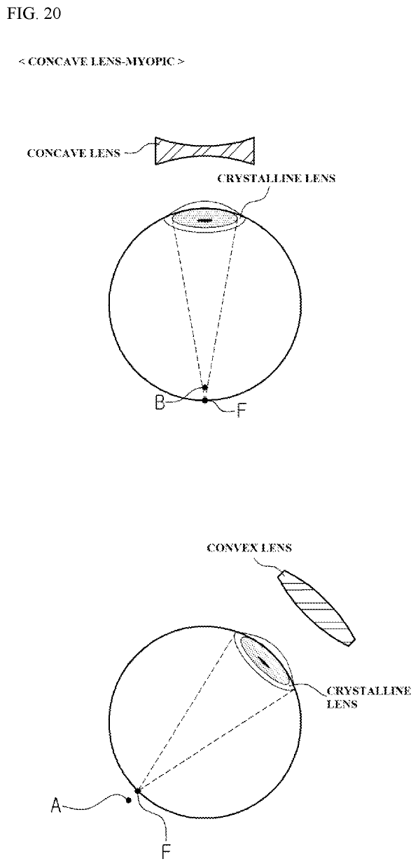

Also, as shown in FIGS. 16B and 20, when the lens member 220 of the front pin hole portion 200 is a concave lens and the lens member 220 of the side pin hole portion 600 is a convex lens to be configured as a myopic type, the concave lens in front allows a focal point of incident light to be formed at a normal focal point F.

On the other hand, when an eyeball is rotated sideward maximally and looks at the convex lens on a side, the eyeball and/or crystalline lens is formed to be slightly longer laterally like a hyperopic eyeball or crystalline lens due to a change in intraocular pressure due to ocular muscle exercise so that a focal point is formed on the outside A of retina. Here, the convex lens of the side pin hole portion 600 pulls a focal point forward to form the focal point at the normal focal point F of retina so that the focal point is accurately formed on the retina in the eyeball to smoothly perform focal movement.

Meanwhile, the side pin hole portion 600 is in connection with the sideward movement portion 700 to vary in position to be appropriate for the wearer. The sideward movement portion 700, which is installed on the sidewall of the body tube 110, moves the side pin hole portion 600 according to manipulation of the wearer. The sideward movement portion may be configured to be equal to the above-described forward movement portion 300.

In detail, the sideward movement portion 700 includes a third rotating shaft 710 rotatably installed on the sidewall of the body tube 110, a second pinion 720 provided on one longitudinal end of the third rotating shaft 710, a side knob 730 provided on the other longitudinal end of the third rotating shaft 710 and exposed outward from the side wall of the body tube 110, and a second rack 620 integrally formed with the side pin hole portion 600 and engaged with the second pinion 720 so as to linearly move the side pin hole portion 600 according to rotation of the second pinion 720.

In the sideward movement portion 700 having the above components, as the wearer rotates the side knob 730, the second pinion 720 is rotated by the third rotating shaft 710 and engaged with the second rack 620 integrally formed with the side pin hole portion 600 to linearly move the sideward movement portion 700 in the side opening portion 112a so as to locate the side pin hole portion 600 at a position desired by the wearer.

Also, as shown in FIG. 10, a stopper 740 is provided on the side knob 730 so as not to allow the side pin hole portion 600 to arbitrarily move when a position of the side pin hole portion 600 is adjusted by the sideward movement portion 700.

The stopper 740 provided on the side knob 730 is screw-fastened to the side knob 730 and an end thereof pressurizes the outer surface 112′ of the sidewall of the body tube 110 according to rotation of the stopper 740 to restrict rotation of the third rotating shaft 710 connected to the side knob 730.

Also, the side knob 730 and the outer surface 112′ of the sidewall of the body tube 110 are graduated to allow the wearer to easily see a movement distance of the side pin hole portion 600 so that the wearer may easily see a position of the side pin hole portion 600 appropriate for the wearer through gradations formed on the side knob 730 and the outer surface 112′ of the sidewall of the body tube 110.

According to the eye exercise device according to the present invention having the above components, since a structure, in which lenses for improving myopia, hyperopia, and astigmatism is provided in a holder, and a structure, in which a plurality of LEDs configured to induce periocular muscles to adequately move are arranged on the forward gaze plate 120, are provided, the gaze of the wearer may be moved according to the blinking LEDs and overall muscles configured to move eyeballs may relax and contract so as to improve visual acuity of the wearer.

In detail, the front pin hole portion uses a principle of pin hole operation. The pin hole operation substantially almost blocks light near a pin hole onto which light is incident to provide only the incident light passing through the pin hole to the eyeballs so as to allow a near or far object to be seen more clearly through artificial restriction on the incident light.

The pin hole operation restricts a variety of phases around the pin hole from flowing in with light around the pin hole. Accordingly, since some of the variety of incident phases are blocked by the pin hole and only a phase of the incident light passing through the pin hole is provided, the object may be more clearly seen. In the pin hole operation, when the eyes are squinted, an amount of light is reduced and a focal point is easily formed on the retina so that the object is seen more clearly.

The above pin hole operation is applied to the block body and the disk of the present invention. In the case of the block body, part of light incident onto an inner circumferential surface is irregularly reflected toward a surface of a groove portion (notch, screw thread, or the like) formed in the inner circumferential surface so as to provide a clearer phase by restricting incident phases included in the part of incident light again.

Here, since the inner circumferential surface of the block body is expanded by the groove portion (notch, screw thread, or the like), the incident light is diffused over the surface of the groove portion (notch, screw thread, or the like) so as to be irregularly reflected on the surface of the groove portion (notch, screw thread, or the like). Also, a linear type inner circumferential surface (path) of the block body and a hole of the disk restrict a light amount of the incident light again to provide a clearer phase.

Also, in the present invention, the lighting device is installed to be adjacent to the forward gaze plate 120 and the wearer's iris reacts to blinking of the lighting device and performs pupil exercise so as to improve visual acuity of the wearer.

Also, in the present invention, positions of the front pin hole portion 200 and the side pin hole portion 600 may vary according to the wearer and be applied to a variety of users while not being limited to a particular wearer so as to assist visual acuity improvement.

Meanwhile, the present invention is not limited to the above-described embodiment and changes and modifications may be made without departing from the scope of the present invention and it should be understood that the changes and modifications are included in the technical concept of the present invention.

For example, at least any one of the forward gaze plate 120 and the sideward gaze plate 630 includes the rear lighting device 130 to perform iris exercise so as to assist in improving eyeball health of the wearer. The rear lighting device 130 will be described on the basis of FIGS. 13, 14, and 17.

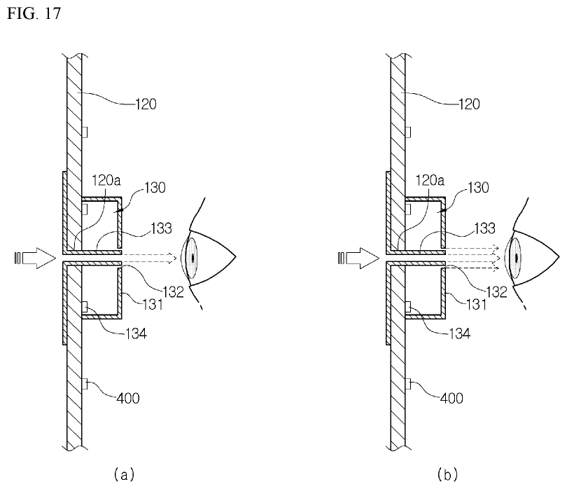

FIGS. 13 and 14 are cross-sectional views illustrating the rear lighting device of the eye exercise device according to the present invention, and FIGS. 17A and 17B are cross-sectional views illustrating eye exercise performed by the rear lighting device in the eye exercise device according to the present invention.

Referring to the drawings, the rear lighting device 130 is provided on a periphery of the through hole 120a and/or 630a formed in the forward gaze plate 120 and/or sideward gaze plate 630 and provides lighting in a blinking state and separate from light passing through the front pin hole 211 and/or side pin hole 611.

As shown in FIG. 17A, the rear lighting device 130 includes the cap 131 surrounding the periphery of the through hole 120a and/or 630a formed in the forward gaze plate 120 and/or the sideward gaze plate 630 to form a space therein and a gaze hole 132 formed in the cap 131 and located collinearly with the through hole 120a and/or 630a formed in the forward gaze plate 120 and/or the sideward gaze plate 630.

The cap 131 provided on the forward gaze plate 120 and/or sideward gaze plate 630 may have a cylindrical shape as shown in FIG. 13 or a semicircular shape as shown in FIG. 14. An LED 134 is provided in the cap 131 and blinks. Here, one or a plurality of such LEDs 134 may be provided in the cap 131. When the plurality of LEDs 134 are provided in the cap 131, the LEDs 134 may operate to have a blinking pattern.

As described above, the LED 134 provided in the cap 131 and blinking may emit light toward the wearer's eyeball through the gaze hole 132 so that the iris may react to light and shade of light emitted through the gaze hole 132 so as to perform exercises assisting in improving eyeball health.

Also, as described above, when the rear lighting device 130 is provided in the forward gaze plate 120, in order to emit light from the front pin hole portion 200 and light from the rear lighting device 130 toward the wearer while being separated from each other, the rear lighting device 130 may include a bush 133 extending from the through hole 120a formed in the forward gaze plate 120 toward the gaze hole 132 and forming a gap from the gaze hole 132 as shown in FIG. 14.

When the bush 133 is provided in the forward gaze plate 120 as described above, as shown in FIG. 17B, rear lighting may be emitted toward the gap between the bush 133 and the gaze hole 132 of the cap 131 and the light emitted from the front pin hole portion 200 and the light emitted from the rear lighting device 130 may be emitted toward the wearer while being separated from each other so as to perform a variety of iris exercises.

The rear lighting device 130 including the bush as described above may operate so as not to form a shade caused by the bush 133 or to allow each of the LEDs 134 to have a blinking pattern when the plurality of LEDs are arranged on the basis of the bush 133 in the cap 131 and rear lighting is emitted toward the gap between the gaze hole 132 and the bush 133.

Also, since the bush 133 disposed to be adjacent to the eyeball guides incident light linearly, the above incident light passing through the block body or the disk may be stably guided to the pupil of the eyeball so that the wearer may easily see a clear phase through the incident light.

In conclusion, since the pin hole, the block body, and the disk restrict peripheral phases incident with the incident light and provide only a filtered phase to the eyeball, a focal point is more easily formed at the retina so as to allow the wearer to actually perform focal point adjustment exercises to improve visual acuity.

Also, as described above, since incident light filtered through the pin hole, the block body, the disk, the bush, and the like is delivered to the pupil and lighting of the rear lighting device 130 blinks, the iris of the wearer is vigorously exercised. That is, since the iris of the wearer is expanded or reduced like an aperture of a camera when the rear lighting device 130 is turned on and a focal point is clearly formed by the above incident light when the rear lighting device 130 is turned off, the eyeball of the wearer is vigorously exercised.

Also, the cap 131 of the rear lighting device 130 provides a pin hole effect through a central hole facing the pupil when the LED 134 of the rear lighting device 130 blinks so as to restrict phases around the hole from being incident due to the rear lighting and to provide a filtered phase to the pupil. Accordingly, the eyeball is more vigorously exercised.

Also, although it has been described above that the dial 330 of the forward movement portion 300 or the side knob 730 of the sideward movement portion 700 is rotationally operated by the wearer, on a case-by-case basis, a driving motor is installed on each of the first rotating shaft 310 and the third rotating shaft 710 so that positions of the front holder 210 and the side holder 610 may be adjusted by an operation of the driving motor.

Also, although it has been described above that the forward movement portion 300 moves the forward gaze plate 120 by operating the driving bevel gear 320 and the driven bevel gear 340, on a case-by-case basis, the forward movement portion 300 may move the forward gaze plate 120 by applying a structure of the second rack 620 and the second pinion 720 like the sideward movement portion 700.

Also, only the lens member may be embedded in the front holder. That is, only the concave lens or convex lens may be embedded in the front holder. On the other hand, only the disk including the through hole may be embedded in the front holder. A plurality of such disks may be embedded in while being overlapped with the through holes communicating with each other. That is, the scattering prevention portion may include only the disks. Also, the lens member may be embedded in the front holder and be overlapped with the disk.

Also, the sideward gaze plate may include at least any one of the above-described indirect lighting device, rear lighting device, and bush.

1. An eye exercise device comprising:
a body tube worn on a face to shield eyes of a wearer; and
a forward gaze plate disposed on one side of the body tube to face the eyes of the wearer,
wherein the forward gaze plate comprises at least any one of a through hole configured to guide light provided from an outside of the body tube to the eyes of the wearer and a light emitting diode (LED) configured to provide lighting light toward the eyes of the wearer.

2. The eye exercise device of claim 1, further comprising a front pin hole portion provided in the forward gaze plate and configured to guide external light to the through hole formed in the forward gaze plate.

3. The eye exercise device of claim 2, wherein the front pin hole portion comprises:
a front holder provided on the forward gaze plate and comprising a space portion therein and a front pin hole through which external light flows; and
a scattering prevention portion provided in the front holder to induce linearity of light passing through the front pin hole.

4. The eye exercise device of claim 3, wherein the scattering prevention portion comprises at least any one of a lighting guide embedded in the front holder and comprising an optical path configured to allow the light flowing in through the front pin hole to pass therethrough or a lens member configured to diffuse or focus the light flowing in through the front pin hole.

5. The eye exercise device of claim 4, wherein the lighting guide is embedded in the front holder and comprises at least any one of a disk and a block body in which the optical path is formed.

6. The eye exercise device of claim 5, wherein the scattering prevention portion comprises a plurality of such lighting guides in which the optical path is provided, and
wherein the lighting guides are embedded in the front holder while the respective optical paths communicate with each other.

7. The eye exercise device of claim 5, wherein at least any one of the lighting guides comprises a single groove portion or a plurality of groove portions formed in an inner circumferential surface, on which the optical path is formed, and configured to prevent sideward-diffused light of incident light flowing in through the optical path from being irregularly reflected so that the diffused light is guided along the optical path.

8. The eye exercise device of claim 5, wherein the lighting guide comprises at least one disk and at least one block body, in which the optical paths are formed, and is embedded in the front holder while the disk and the block body are overlapped with each other, and
wherein the block body and the disk are embedded in the front holder while the respective optical paths communicate with each other.

9. The eye exercise device of claim 4, wherein the lens member is embedded in the front holder while being overlapped with the lighting guide.

10. The eye exercise device of claim 4, further comprising an indirect lighting device configured to contract or expand a pupil of the wearer by providing blinking lighting light separate from light passing through the front pin hole to at least one of the optical paths of the lighting guides configured to allow the light flowing in through an inside of the front holder or the front pin hole to pass therethrough.

11. The eye exercise device of claim 1, wherein the forward gaze plate comprises a main blinking module in which a plurality of such LEDs are spaced apart from each other and which is provided in an inner surface facing the wearer and configured to blink the LEDs in a pattern set to induce a gaze of the wearer to sequentially provide lighting light to the eyes of the wearer.

12. The eye exercise device of claim 11, wherein the forward gaze plate comprises a through hole formed in a center of the inner surface and guides light provided from an outside of the body tube to the eyes of the wearer separately from the LEDs, and
wherein the LEDs are arranged radially or on all sides on the basis of the through hole and provide lighting light based on the light from the outside which is emitted through the through hole.

13. The eye exercise device of claim 1, wherein a pair of such forward gaze plates are provided, and
wherein the body tube further comprises a forward movement portion configured to move the pair of forward gaze plates to dispose the LEDs or the through holes at positions corresponding to the eyes of the wearer.

14. The eye exercise device of claim 13, wherein the forward movement portion comprises a rack and pinion gear or a bevel gear to move the forward gaze plates and moves the forward gaze plates as the forward gaze plates are connected to some components and in connection therewith.

15. The eye exercise device of claim 1, further comprising a sideward gaze plate installed on a side of the forward gaze plate and comprising at least any one of an LED configured to provide lighting light toward the eyes of the wearer and a through hole configured to guide light from an outside of the body tube to the eyes of the wearer to induce a gaze of the wearer sideward.

16. The eye exercise device of claim 15, wherein the sideward gaze plate further comprises a side pin hole portion configured to guide the light provided from the outside of the body tube to the through hole.

17. The eye exercise device of claim 16, wherein the side pin hole portion comprises:
a side holder comprising a side pin hole, through which the light provided from the outside of the body tube flows in, and a space portion provided therein; and
a scattering prevention portion provided on the side holder and configured to induce linearity of the light passing through the side pin hole.

18. The eye exercise device of claim 17, wherein the scattering prevention portion comprises at least any one of a lighting guide embedded in the side holder and comprising an optical path configured to allow the light flowing in through the side pin hole to pass therethrough or a lens member configured to diffuse or focus the light flowing in through the side pin hole.

19. The eye exercise device of claim 15, wherein the sideward gaze plate comprises an auxiliary blinking module in which a plurality of such LEDs are spaced apart from each other and which is provided in an inner surface facing the wearer and configured to blink the LEDs in a pattern set to induce a gaze of the wearer sideward to sequentially provide lighting light to a side of the wearer.

20. The eye exercise device of claim 19, wherein the sideward gaze plate comprises a through hole formed in a center of the inner surface and guides light provided from an outside of the body tube to the eyes of the wearer separately from the LEDs, and
wherein the LEDs are arranged radially or on all sides on the basis of the through hole and provide lighting light based on the light from the outside which is emitted through the through hole.

21. The eye exercise device of claim 15, wherein the sideward gaze plate is moved along a sidewall of the body tube by a sideward movement portion provided in the body tube.

22. The eye exercise device of claim 15, wherein the sideward gaze plates are formed at an angle tilted from the forward gaze plate and gradually move farther away from each other from a front of the body tube toward the wearer.

23. The eye exercise device of claim 1, further comprising a rear lighting device configured to provide lighting separate from the LED of the forward gaze plate to a rear of the forward gaze plate.

24. The eye exercise device of claim 23, wherein the rear lighting device comprises:
a rear LED configured to operate separately from the LED of the forward gaze plate and to emit rear lighting to the rear of the forward gaze plate;
a cap configured to shade the rear LED; and
a gaze hole formed in the cap to pass the lighting of the rear LED therethrough.

25. The eye exercise device of claim 1, further comprising a bush installed in the through hole of the forward gaze plate to guide light flowing in through the through hole straight toward a rear of the forward gaze plate.
